(19) United States
(12) Patent Application Publication
(10) Pub. No.: US 2014/0352745 A1
(43) Pub. Date: Dec. 4, 2014

(54) Cleaning and Disinfecting Device

(71) Applicant: Meiko Maschinenbau GmbH & Co. KG, Offenburg (DE)

(72) Inventor: Ingo Wiegand, Buhlertal (DE)

(73) Assignee: Meiko Maschinenbau GmbH & Co. KG, Offenburg (DE)

(21) Appl. No.: 14/164,432

(22) Filed: Jan. 27, 2014

Related U.S. Application Data
(60) Provisional application No. 61/829,312, filed on May 31, 2013.

(51) Int. Cl. E03D 9/00; B08B 9/08; A47K 11/00

(52) U.S. Cl. CPC E03D 9/00; A47K 11/00; B08B 9/08
USPC 134/57 R; 134/113

(57) ABSTRACT

The invention relates to a cleaning and disinfecting device (10). The cleaning and disinfecting device (10) comprises a wash chamber (12), which is closed by a wash chamber door (14) and which can be actuated by means of a drive (26). A pivoting path or a pivoting area (24) of the wash chamber door (14) is monitored by at least one sensor (22).

### TECHNICAL FIELD

The invention relates to a cleaning and disinfecting device which is used in hospitals, care homes and the like for cleaning containers.

### PRIOR ART

Cleaning and disinfecting devices are used in particular in hospitals and care homes for persons needing care and are used in particular for treating containers which are intended to receive human excretions. The cleaning and disinfecting devices under discussion here are in particular those which have a wash chamber door which is equipped with an automatic drive and is actuated by the latter and opens or closes the access to a wash chamber, in which the containers under discussion are cleaned. There is an extremely wide range of designs of cleaning and disinfecting devices, in particular with regard to the access to the wash chamber. One possible design variant is provided by a folding door, which is located on a device front of the cleaning and disinfecting device, which is provided with a pivot axis, in particular parallel to its lower edge, and which, in order to be opened, is pivoted up by an operator, toward the latter. This design variant exists in an extremely wide range of different variants, which are substantially actuated manually by the operator. Furthermore, there are variants in which the wash chamber door closing the access to the wash chamber can be opened or closed by an automatic drive, in particular by means of an electric motor. A drive of this type actuating the wash chamber door is activated, for example, by a foot switch, which is actuated by the operator, or a non-contact proximity switch which, for example, reacts to the approach of a part of a body of the operator, or by the machine control system of the cleaning and disinfecting device at the end of a program sequence. The advantage of such design variants is based in particular on the fact that the operator is able to load the cleaning and disinfecting device, in particular the wash chamber of the latter, without having to actuate the wash chamber door closing the wash chamber with their hands. As a result, the ease of operation and hygienic safety of such cleaning and disinfecting devices is improved significantly.

Furthermore, there exist design variants of cleaning and disinfecting devices having a wash chamber door on the device front which, for example, are installed in a wet cell, for example in the surroundings of a patient room of a hospital. Such cleaning and disinfecting devices can, for example, be free-standing in the wet cell or let flush into a wall of the wet cell. A WC, for example, can be fitted directly to such cleaning and disinfecting devices in the wet cell. This arrangement is relatively widespread and reduces the expenditure on installation significantly; furthermore, the expenditure on apparatus is minimized and, as a result, the space required for such a cleaning and disinfecting automat for use in sanitary equipment is usually optimised.

If the wet cell is equipped in this way, for example, a patient who is not bedridden can readily use the WC. On the other hand, if the patient is bedridden, the cleaning and disinfecting device free-standing in the wet cell or installed flush in a wall can be used for cleaning and disinfecting the bedpans receiving the human excretions. If a plurality of patient beds is assigned to such a wet cell, then both types of use can be applied simultaneously.

Cleaning and disinfecting devices of this type, in particular those described above, have the disadvantage, in particular in the case of automatic actuation of the wash chamber door and a WC mounted in front, that it is possible for a collision situation to occur in the event of automatic door opening of the wash chamber door. For example, a wash chamber door which is actuated automatically by the machine control system at the end of a cleaning program can collide with a WC lid covering a WC bowl of a WC which has been lifted upright. One possible consequence can consist in these two elements jamming each other and, in the worst case, the cleaning and disinfecting device can be damaged. Furthermore, it can be imagined that a person who is currently using the WC can be injured by an automatically opening wash chamber door. For the case in which the cleaning and disinfecting device installed in a wet cell does not have a WC fitted in front but is equipped with automatic wash chamber door actuation, a carer or another person in the room could also collide with an automatically opening wash chamber door. Furthermore, there is a possibility that, for example, a wheelchair or another larger care aid, such as a bath lift, is located in the pivoting area of an automatically opening wash chamber door of a cleaning and disinfecting device, and it is possible for a collision to occur in the event of automatic opening of the wash chamber door.

If there are very confined space conditions in the wet cell, it is further possible to imagine that an open room door, the door of a shower cubicle or the door of a piece of furniture or the like could collide with an automatically opening wash chamber door.

### SUMMARY OF THE INVENTION

The present invention is based on the object of providing a cleaning and disinfecting device which avoids the disadvantages of the design variants outlined above known from the prior art.

According to the invention, a cleaning and disinfecting device is proposed which is either erected free-standing in a wet cell or is let into a wall bounding a wet cell, and which has an automatically actuated wash chamber door and is to be equipped with at least one sensor which monitors the room or a pivoting path which is required by an automatically actuated wash chamber door when the latter is moved from its closed position into a final opened position and vice versa.

In particular, the at least one sensor proposed in accordance with the invention is incorporated into the control system of the cleaning and disinfecting device or connected to said control system. If the at least one sensor detects a person or an object in the range of movement of the wash chamber door, for example, then the movement of the wash chamber door is interrupted or the movement is prevented from the start, i.e. is even not initiated at all.

If such a case is present, there are a number of reaction possibilities of the cleaning and disinfecting device to react thereto. For example, there is the possibility of outputting a message on a display of the control system of the cleaning and disinfecting device, which requires an operator to check the range of movement, in particular the pivoting area of the automatically actuated wash chamber door, and to re-activate the opening procedure of the wash chamber door. A further conceivable reaction of the cleaning and disinfecting device or of the control system of the latter consists in checking the pivoting area of the wash chamber door continuously by means of the at least one sensor and, if the latter enables it, initiating an automatic opening of the wash chamber door closing the wash chamber. There is a possibility of outputting the above warning message alternatively or in addition to the optical display on the display, including in the form of an acoustic signal.

In an advantageous design variant of the cleaning and disinfecting device proposed in accordance with the invention, the at least one sensor can be let into the device front of the cleaning and disinfecting device or it can be fitted above the cleaning and disinfecting device, for example in the ceiling area of the wet cell or of the room in which the cleaning and disinfecting device is erected. Other arrangements which permit monitoring of the pivoting area of an automatically actuated wash chamber door are likewise conceivable, for example an arrangement of the sensor on a wall of the room.

The at least one sensor proposed according to the invention on the cleaning and disinfecting device can, for example, be implemented as an optical proximity switch, for example as a light sensor, which operates either in the visible light spectrum or in an invisible light spectrum, such as in the infrared range. Furthermore, there is the possibility also of using other measuring principles or measuring methods than a proximity switch or light sensor for the at least one sensor as sensing means; furthermore, there is also the possibility of using at least one sensor which uses a combination of several different measuring methods. By way of example, mention should be made here of passive infrared sensors, ultrasound sensors, microwave sensors and the like.

In addition or as an alternative to the above list of sensors, monitoring of the pivoting area of the wash chamber door can be carried out by means of a camera system, for example a video camera and/or the like, and corresponding image evaluation, for example, in the control system and, in this way, the desired functionality can be provided. Furthermore, there is a possibility of not using just one sensor for the monitoring of the pivoting area of the wash chamber door but also of using two or more sensors which operate in accordance with different functional principles. These several sensors can be linked to different signal inputs in the control system of the cleaning and disinfecting device. Furthermore, the control system of the cleaning and disinfecting device can be implemented in such a way that there is an additional signal input on said control system for a camera system for monitoring the pivoting area. The camera system then consists, for example, at least of a camera itself, hardware for image processing and signal output, and software for image processing and image evaluation. The at least three named components of the camera system can be combined in one structural unit. The camera can either record images continuously, for example a film, or individual images progressively. An evaluation for disruptive bodies and/or objects in the space to be monitored can be implemented with both recording methods.

The different signal inputs can be represented in the control system by a “watchdog” function. This is understood generally, amongst other things, to mean a component of a system which monitors the function of other components. If a possible malfunction is detected, then this is either signaled in accordance with the system convention or a suitable jump instruction is initiated, which rectifies the problem present.

The invention will be described more extensively below by using the drawing, in which:

FIG. 1 shows an oblique view of a cleaning and disinfecting device according to the invention installed in a wall with front-mounted WC,

FIG. 2 shows a front view of a cleaning and disinfecting device according to the illustration in FIG. 1, which is incorporated in a wall of a wet cell with a wash chamber closed by a wash chamber door,

FIG. 3 shows a section through the design variant of the cleaning and disinfecting device according to FIG. 2 with waste water and fresh water piping, and

FIG. 4 shows a section through a cleaning and disinfecting device according to the invention installed in a wall with front-mounted WC, with opened wash chamber door, and also the illustration of the pivoting area of a wash chamber door closing the wash chamber of the cleaning and disinfecting device.

### DESCRIPTION OF THE DRAWINGS

The illustration according to FIG. 1 reveals a perspective view of a cleaning and disinfecting device let into a wall with WC or bowl of a WC mounted in front.

A cleaning and disinfecting device 10 comprises a wash chamber 12 closed by a wash chamber door 14 in the illustration according to FIG. 1. In the wash chamber 12 closed by the wash chamber door 14, containers 16 containing human excretions are cleaned and disinfected. The cleaning and disinfecting device 10 illustrated in FIG. 1 is let into a wall 18. On the wall 18, which, for example, bounds a wet cell of a hospital or a care home or the like, there is fixed a WC bowl 20 of a WC, which is closed by a WC lid 21.

From the illustration according to FIG. 1, it emerges that there is at least one sensor 22 in the lower region of a device front 32 of the cleaning and disinfecting device 10. This sensor can be, for example, a proximity switch, what is known as a light sensor, which operates in the visible light spectrum or in the invisible light spectrum, for example in the infrared range. The at least one sensor 22 can also be a passive infrared sensor, an ultrasonic sensor or a microwave sensor and/or the like. Furthermore, the sensor 22 can be a camera system. The camera system then comprises at least a camera itself, hardware and software for image processing and image evaluation. The at least three named components of the camera system can be combined in one structural unit. The camera can either record images continuously, for example a film, or individual images progressively. An evaluation for disruptive bodies and/or objects in the space to be monitored can be implemented with both recording methods.

The at least one sensor 22 monitors whether the WC bowl 20 is being used by a person or whether the WC lid 21 has been folded up or not. As long as the WC is being used, the sensor 22 stops the wash chamber door 14 being opened. Furthermore, in the design variant according to FIG. 1, the at least one sensor 22 integrated into the device front 32 of the cleaning and disinfecting device 10 can be arranged in such a way that the said sensor monitors a pivoting area or a space 24 swept over by the wash chamber door 14, which can be actuated by means of an electric drive 26, during the transition from its closed position 28 into an open position 30. In the illustration according to FIG. 1, the drive which moves the wash chamber door 14 from its closed position 28 into its open position 30 is indicated only schematically as an electric drive 26. The wash chamber door 14 is pivoted by the drive 26, for example constructed as an electric motor, about a pivot axis 34 which extends in the horizontal direction parallel to the lower edge of the automatically actuatable wash chamber door 14.

The illustration according to FIG. 1 further reveals that an operating area 50 is integrated into the device front 32 of the cleaning and disinfecting device, as is a display 52.

A control system 40 of the cleaning and disinfecting device, with which a program sequence for cleaning and disinfecting containers 16 containing human excretions within the wash chamber 12 can be controlled, is not illustrated in FIG. 1.

FIG. 2 shows a front view of a cleaning and disinfecting device incorporated in a wall.

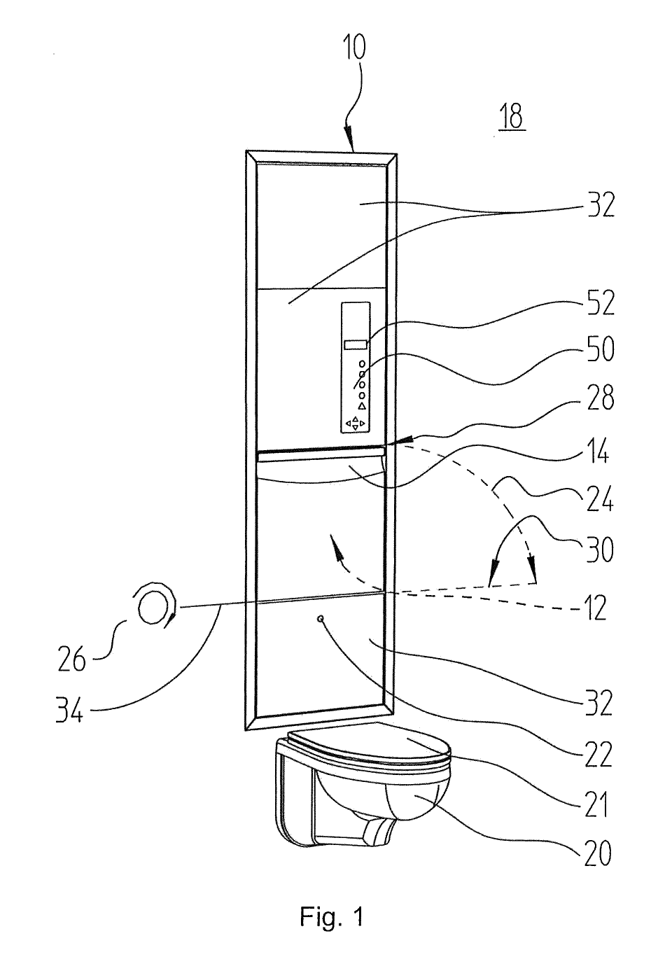

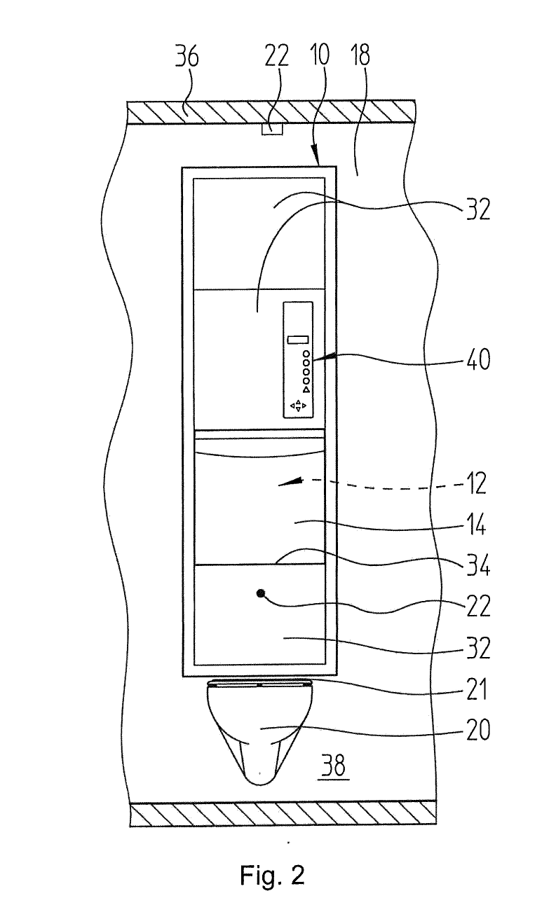

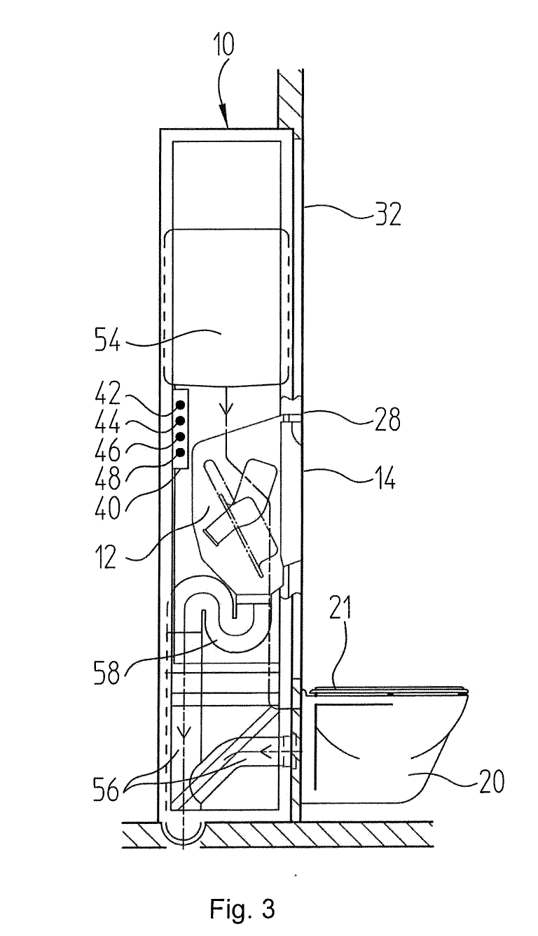

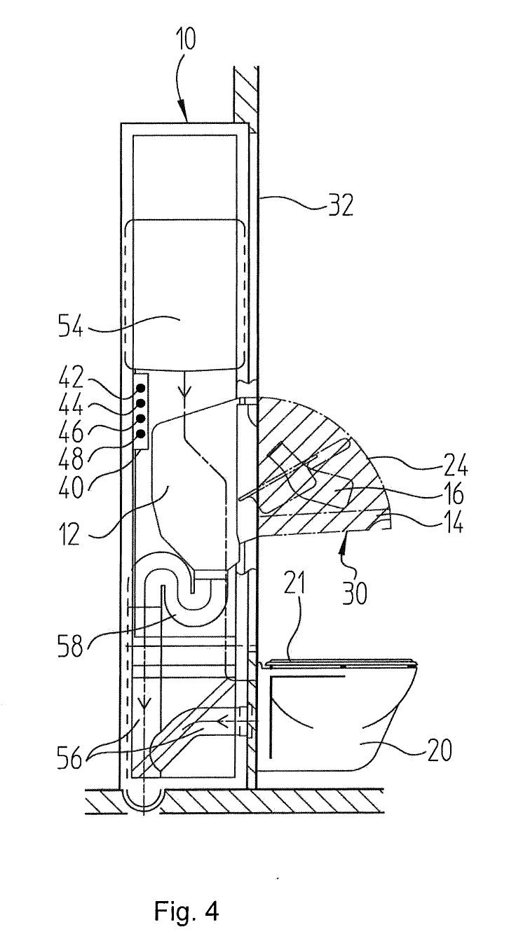

From the illustration according to FIG. 2, it emerges that the wash chamber 12 is arranged behind the wash chamber door 14 illustrated in its closed position 28 in FIG. 2. In the view according to FIG. 2, which comprises a cleaning and disinfecting device let into the wall 18, the wash chamber 12 is closed by the wash chamber door 14. The WC bowl 20, which is closed by the WC lid 21, is fixed to the wall 18. Reference symbol 38 indicates the room in the boundary wall 18 of which the cleaning and disinfecting device 10 is installed. In the case illustrated in FIG. 2, for example the at least one sensor 22 is let into the ceiling 36 which bounds the room 38 at the top. Behind the operating area 50 and/or the display 52 there is a control system 40, which has several signal inputs 42, 44, 46 and 48 (cf. illustration according to FIG. 3).

In the sectional illustration according to FIG. 3, the design variant of the cleaning and disinfecting device 10 which is reproduced in front view in FIG. 2 is illustrated. The wash chamber door 14 is illustrated in its closed position 28. The control system 40, which is integrated into the cleaning and disinfecting device 10, comprises the said signal inputs 42, 44 and 46 and a signal input 48 to which a camera system for monitoring the pivoting area and/or the space 24 can be connected. The cleaning and disinfecting device 10 according to the sectional illustration in FIG. 3 comprises a flushing water tank 54, in which, for example, the water needed to flush the WC is stored. Reference symbol 56 designates waste water lines, which lead the water both from the wash chamber 12 of the cleaning and disinfecting device 10 and the flushing water from the WC 20 away into a sewer.

It can be gathered from the illustration according to FIG. 4 that, underneath the wash chamber 12 that can be closed by the wash chamber door 14, there extends a siphon bend 58, which merges into the waste water line 56.

FIG. 4 shows that the automatically actuatable wash chamber door 14 has moved from its closed position 28 into its open position 30. The area 24 shown hatched in FIG. 4 is the pivoting area of the automatically actuated wash chamber door 14, which is swept over by the latter when the automatically actuated wash chamber door 14 is moved from its closed position 28 into its open position 30. In order to avoid risks of injury or damage occurring in the process, one or more sensors can be installed in the boundary walls 18 of the room 38 in which the cleaning and disinfecting device 10 is incorporated, either free-standing or in one of the boundary walls 18, which sensors function in accordance with one and the same or else in accordance with different measuring principles.

In the illustration according to FIG. 4, the pivoting area 24 of the automatically actuatable wash chamber door 14 is illustrated hatched. Indicated schematically in the hatched area is a container 16 which, in this case, is located in a holder on the wash chamber door outside the wash chamber 12 and, together with the holder, when the wash chamber door 14 is actuated, can be introduced by the operator into the wash chamber 12 of the cleaning and disinfecting device 10.

Instead of an individual sensor 22 for monitoring the pivoting area 24 of the automatically actuated wash chamber door 14, use can also be made of several sensors 22, which operate in accordance with different measuring principles. Thus, monitoring of the pivoting area 24 of the wash chamber door 14 in any position is ensured, so that collisions with and risks of injury to, for example, the patient using the WC 20 are ruled out, and risks of collision with doors, with people who are in the room 38 and the like are minimized.

Furthermore, it is also possible to connect a camera system, with which the room 38 can be monitored completely, to the signal input 48 of the control system 40 (cf. illustration according to FIG. 3). With the aid of this camera system, which can be connected to the signal input 48 of the control system 40, it is possible firstly just to monitor the pivoting area 24 of the wash chamber door 14 as to whether persons or objects are located therein. Secondly, furthermore, it is also possible to integrate into the camera system emergency functions which, for example, detect unconsciousness of a patient, so that, for example via the control system 40 and a link, not specifically illustrated, to a central control desk, help can quickly be requested and given.

### LIST OF REFERENCE SYMBOLS

10 Cleaning and disinfecting device
12 Wash chamber
14 Wash chamber door
16 Container
18 Wall
20 WC bowl
21 WC lid
22 Sensor
24 Pivoting area, space to be monitored
26 Drive
28 Closed position
30 Open position
32 Device front
34 Pivot axis
36 Room ceiling
38 Room
40 Control system
42 First signal input
44 Second signal input
46 Third signal input
48 Signal input for camera system
50 Operating area
52 Display
54 Water tank
56 Waste water line
58 Siphon bend

1. Cleaning and disinfecting device (10), having a wash chamber (12) that can be closed by a wash chamber door (14), in which containers (16) containing human excretions are cleaned, the wash chamber door (14) being moved by means of a drive (26), characterized in that at least one sensor (22) is provided, which monitors the space (24) in front of the cleaning and disinfecting device (10).

2. Cleaning and disinfecting device according to claim 1, characterized in that the cleaning and disinfecting device (10) comprises a WC (20, 21), the sensor detecting a use of the WC (20, 21).

3. Cleaning and disinfecting device according to claim 1, characterized in that the at least one sensor (22) monitors a pivoting area (24) which is swept over by the moved wash chamber door (14) when the latter is moved from its closed position (28) into its opened position (30) and vice versa.

4. Cleaning and disinfecting device according to one of the preceding claims, characterized in that the at least one sensor (22) is let into a device front (32) of the cleaning and disinfecting device (10).

5. Cleaning and disinfecting device according to one of the preceding claims, characterized in that the at least one sensor (22) is let into the device front (32) underneath a pivot axis (34) of the wash chamber door (14) of the cleaning and disinfecting device (10).

6. Cleaning and disinfecting device according to one of the preceding claims, characterized in that the at least one sensor (22) is arranged in a ceiling (36) and/or in a wall (18) of a room (38) in which the cleaning and disinfecting device (10) is located.

7. Cleaning and disinfecting device according to one of the preceding claims, characterized in that the at least one sensor (22) is an optical proximity switch.

8. Cleaning and disinfecting device according to the preceding claim, characterized in that the at least one optical proximity switch (22) operates in the visible or in the invisible light spectrum, in particular in the infrared spectrum.

9. Cleaning and disinfecting device according to one of the preceding claims, characterized in that the at least one sensor (22) is implemented as a passive infrared sensor or as an ultrasonic sensor or as a microwave sensor or as a sensor which combines and unites different measuring methods.

10. Cleaning and disinfecting device according to one of the preceding claims, characterized in that the cleaning and disinfecting device (10) comprises a control system (40) which comprises several signal inputs (42, 44, 46) for one or more sensors.

11. Cleaning and disinfecting device according to one of the preceding claims, characterized in that the control system (40) has at least one signal input (48) for a camera system.

12. Cleaning and disinfecting device according to one of the preceding claims, characterized in that the control system (40) checks the space (24) to be monitored with the aid of the at least one sensor (22) and, if a possible collision situation is detected, prevents or interrupts an opening of the wash chamber door (14), and an occurrence of a collision situation is displayed in the display (52).

13. Cleaning and disinfecting device according to one of the preceding claims, characterized in that the control system (40) continuously checks the space (24) to be monitored with the aid of the at least one sensor (22) and, following the elimination of the collision situation, automatically initiates an opening of the wash chamber door (14).
